(19) United States
(12) Patent Application Publication
(10) Pub. No.: US 2021/0251946 A1
(43) Pub. Date: Aug. 19, 2021

(54) CANNABINOID-CONTAINING EXTRACTS, FORMULATIONS, AND USES THEREOF FOR TREATING AMYOTROPHIC LATERAL SCLEROSIS

(71) Applicant: GREEN RUSH SCIENCE, LLC, Lake Oswego, OR (US)

(72) Inventor: JOSEPH CHARLES GYDOSH, Lake Oswego, OR (US)

(21) Appl. No.: 17/168,524

(22) Filed: Feb. 5, 2021

Related U.S. Application Data
(60) Provisional application No. 62/970,396, filed on Feb. 5, 2020.

(51) Int. Cl. A61K 31/352; A61K 31/05; A61K 9/127; A61K 9/00; A61K 9/70; A61K 9/107

(52) U.S. Cl. CPC A61K 31/352; A61K 31/05; A61K 9/1271; A61K 9/1075; A61K 9/7023; A61K 9/0078; A61K 9/006

(57) ABSTRACT

The present disclosure provides a pharmaceutical composition for preventing, treating, slowing the progression of, and/or delaying the onset of ALS comprising a functional cannabinoid product and a delivery agent.

### BACKGROUND

Amyotrophic lateral sclerosis (“ALS”) is a group of rare neurological diseases, also known as motor neurone disease (“MND”) or Lou Gehrig's disease, that mainly involves the gradual deterioration or degeneration and death of motor neurons. Motor neurons are nerve cells forming part of a pathway along which impulses pass from the brain or spinal cord to a muscle or gland and are responsible for controlling voluntary muscle movement.

ALS is typically characterized by stiff muscles, muscle twitching, and gradually worsening weakness due to muscles decreasing in size. ALS typically begins with weakness in the arms or legs, difficulty speaking, and/or difficulty swallowing. Most people affected by ALS experience pain and a significant percentage develop difficulties with thinking and behavior. Most of those who suffer from ALS eventually lose the ability to walk, use their hands, speak, swallow, and breathe. In 2016 the Centers for Disease Control and Prevention estimated that between 14,000-15,000 Americans have ALS. No cure for ALS is presently known. Rather, the goal of treatment is to improve symptoms and slow disease progression.

Although the mechanisms that underlie ALS are not precisely defined, it is believed that ALS has a multifactorial etiology, where environmental factors and genetics contribute to triggering its progressive pathology. It is also believed that various mechanisms, including mitochondrial dysfunction, protein aggregation, oxidative stress, excessive glutamate activity, inflammation, and/or apoptosis, are involved in ALS pathogenesis insofar as these conditions lead to motor neuron cell death in the brain and spinal cord. See, e.g., Zarei et al. A comprehensive review of amyotrophic lateral sclerosis. Surg Neurol Int. 2015; 6:171.

To date, the only established therapy for ALS is the glutamate-antagonist riluzole, which is believed to work by inhibiting the presynaptic release of glutamate. Riluzole has limited therapeutic efficacy and has only been shown to moderately prolong patient survival. Non-invasive ventilation may result in improved quality and length of life in patients with ALS. Mechanical ventilation and/or use of a feeding tube can also prolong survival but does not stop disease progression. Accordingly, new therapeutic approaches for improving motor impairment, delaying neurodegeneration, and relieving the symptoms of ALS are needed.

Cannabis is a genus of flowering plants that includes Cannabis sativa, Cannabis indica, and Cannabis ruderalis. Hemp is a type of Cannabis sativa defined as having no more than three-tenths of one percent (i.e., 0.3%) concentration of Δ9-tetrahydrocannabinol (“Δ9-THC”). Cannabis sativa contains over 500 compounds, including over 100 cannabinoids. Fischedick et al. A qualitative and quantitative HPTLC densitometry method for the analysis of cannabinoids in Cannabis sativa L. Phytochem Anal 20:421-426. Cannabinoids are a group of compounds that act on cannabinoid receptors within the endocannabinoid system. Cannabinoids include Δ9-tetrahydrocannabinol (“Δ9-THC”), Δ8-tetrahydrocannabinol (“Δ8-THC”), tetrahydrocannabinolic acid (“THCa”), cannabidiol (“CBD”), cannabidiolic acid (“CBDa”), cannabigerol (“CBG”), cannabigerolic acid (“CBGa”), cannabidivarin (“CBDV”), cannabidivarinic acid (“CBDVa”), cannabigerol (“CBG”), cannabigerolic acid (“CBGa”), cannabinol (“CBN”), cannabinolic acid (“CBNa”), cannabichromene (“CBC”), cannabichromenic acid (“CBCa”), cannabicyclol acid (“CBLa”), tetrahydrocannabivarin (“THCV”), and tetrahydrocannabivarinic acid (“THCVa”). Cannabinoids that are produced endogenously are referred to as endocannabinoids. Cannabnoids can also be chemically or synthetically synthesized. Wiley et al. Hijacking of Basic Research: The Case of Synthetic Cannabinoids, Methods Rep RTI Press. 2011. Cannabinoids that are plant-synthesized are referred to as phytocannabinoids. Phytocannabinoids can be extracted and/or isolated from the Cannabis plant. Phytocannabinoids can also be extracted in combination with other compounds and molecules that are present in the Cannabis plant, such as terpenes. Terpenes are unsaturated hydrocarbons found in the essential oils of plants. Cannabis terpenes include α-Pinene, Camphene, Sabinene, (−) -β-Pinene, β-Myrcene, α-Phelandrene, δ-3-Carene, α-Terpinene, ρ-Isopropyltoluene, δ-Limonene, Eucalyptol, Ocimene 1, Ocimene 2, γ-Terpinene, Sabinene Hydrate, Fenchone, Terpinolene, Linalool, Endo-Fenchyl Alcohol, Camphor, (−)-Isopulegol, Isoborneol, Borneol, Hexahydro Thymol, α-Terpineol, γ-Terpineol, Nerol, Pulegone, Geraniol, Farnesene, β-Caryophyllene, α-Humulene, Valencene, Geranyl Acetate, cis-Nerolidol, β-Nerolidol, trans-Nerolidol, Caryophyllene Oxide, (−)-Guaiol, Cedrol, and (−)-α-Bisabolol. The amount of each compound that is ultimately present in a Cannabis extract can vary depending on the starting material, the extraction technique that is employed, and/or any processing or isolation steps that follow the extraction process.

One way in which cannabinoids exhibit biological activity is by binding cannabinoid (“CB”) receptors, which are receptors in the G-protein coupled receptor (“GPCR”) family. There are in general two types of CB receptors, CB₁ and CB₂, but other types are thought to exist in the central and peripheral nervous systems. See, e.g., Pertwee et al. Cannabinoid receptors and their ligands: beyond CB(1) and CB(2). Pharmacol Rev 2010; 62:588-631. CB₁ receptors are expressed primarily on neurons and glial cells in the brain. CB₂ receptors are found predominantly in the cells of immune system. Following cannabinoid agonist binding and signaling, these receptors exert an inhibitory effect on adenylate cyclaseactivity, activation of mitogen-activated protein kinase, regulation of calcium and potassium channels, and various other signal transduction pathways. See, e.g., Munro et al. Molecular characterization of a peripheral receptor for cannabinoids. Nature 1993; 365:61-65.

Studies have shown that cannabinoids can inhibit the release of pro-inflammatory cytokines and chemokines, thereby suppressing inflammatory responses. See, e.g., Velayudhan et al. Therapeutic potential of cannabinoids in neurodegenerative disorders: a selective review. Curr Pharm Des 2014; 20:2218-2230. Cannabinoids have also shown ability to inhibit oxidative and nitrosative stress, modulate the expression of inducibile nitric oxide synthase, and reduce production of reactive oxygen species (“ROS”). See, e.g., Velayudhan et al. Cannabinoids have also been found to exert anti-glutamatergic action by inhibiting glutamate release and enhancing the effect of the inhibitory neurotransmitter gamma-aminobutyric acid (“GABA”), thereby having neuroprotective effects. See, e.g., Croxford. Therapeutic potential of cannabinoids in CNS disease. CNS Drugs 2003; 17:179-202.

In this context, cannabinoids have demonstrated antioxidant, anti-inflammatory, and neuroprotective effects in pre-clinical animal models of ALS, where a significant delay in disease progression was found when the CB₁/CB₂ receptor agonist WIN 55,212-2 was administered intraperitoneally to ALS hSOD(G93A) mice beginning after onset of motor impairment and tremor (at 90 days old). See, e.g., Bilsland et al. Increasing cannabinoid levels by pharmacological and genetic manipulation delay disease progression in SOD1 mice. FASEB J 2006; 20:1003-1005. In this mouse model, genetic ablation of the fatty acid amide hydrolase (“FAAH”) enzyme, which results in raised levels of the endocannabinoid anandamide, prevented the appearance of disease signs in 90-day-old to ALS hSOD(G93A) mice. The neuroprotective effects of cannabinoids in this model were generally ascribed to a decrease of microglial activation, presynaptic glutamate release, and formation of ROS.

It has also been demonstrated that mRNA receptor binding and function of CB₂ receptors are dramatically and selectively up-regulated in the spinal cords of ALS hSOD(G93A) mice in a temporal pattern that parallels disease progression. Shoemaker et al. The CB₂ cannabinoid agonist AM-1241 prolongs survival in a transgenic mouse model of amyotrophic lateral sclerosis when initiated at symptom onset. J Neurochem 2007; 101:87-98. It was found that daily intraperitoneal administration of the selective CB₂ agonist, AM-1241, initiated after disease onset in ALS hSOD(G93A) mice, delayed motor impairment and increased survival by 56%. In this mouse model, the beneficial effects of cannabinoids were generally ascribed to the CB₂ receptor-mediated suppression of microglial/macrophage activation in the spinal cords of symptomatic G93A mice and due to selective up-regulation of CB₂ receptors in spinal cords as a compensatory and protective measure in the mice.

There has been further evidence of neuroprotective effects using a mixture of two extracts in approximately a 1:1 ratio (2.7 mg of Δ9-THC and 2.5 mg of CBD) in a drug known as Sativex®. Moreno-Martet et al. Changes in endocannabinoid receptors and enzymes in the spinal cord of SOD1(G93A) transgenic mice and evaluation of a Sativex( )-like combination of phytocannabinoids: interest for future therapies in amyotrophic lateral sclerosis. CNS Neurosci Ther 2014; 20:809-815. Sativex® was investigated by using ALS hSOD(G93A) transgenic mice and the extract mixture was found to be effective in delaying ALS progression in the early stages of disease as well as in animal survival, although efficacy decreased during progression of disease. It was also demonstrated that changes occur in endocannabinoid signaling, particularly a marked up-regulation of CB₂ receptors in SOD(G93A) transgenic mice together with an increase of N-acyl phosphatidylethanolamine phospholipase D (“NAPE-PLD”) enzyme responsible for the generation of anandamide (N-arachidonoylethanolamine), the ligand of cannabinoid and vanilloid receptors.

### BRIEF SUMMARY

In accordance with an aspect of the present disclosure, there is provided a pharmaceutical composition for preventing, treating, slowing the progression of, and/or delaying the onset of ALS comprising a functional cannabinoid product and a delivery agent, the functional cannabinoid product comprising at least 85% cannabinoids including one or more of Δ9-THC, THCa, THCV, THCVa, CBD, CBDa, CBN, CBNa, CBG, CBGa, CBC, CBCa and at least 10% non-cannabinoids including one or more of β-Myrcene, δ-Limonene, Linalool, β-Caryophyllene, α-Humulene, β-Nerolidol, (−)-Guaiol, (−)-α-Bisabolol, Terpinolene, Nerolidol, Guaiol, Geraniol, Ocimene 2, Endo-Fenchyl Alcohol, α-Terpineol, Geranyl Acetate, cis-Nerolidol, α-Pinene, (−) -β-Pinene, Farnesene, Valencene, trans-Nerolidol, Caryophyllene Oxide. In some embodiments, the ratio between Δ9-THC combined with THCa and CBD combined with CBDa is between 20:1 and 1:1. In some embodiments, the ratio between Δ9-THC combined with THCa and CBG combined with CBGa is between 60:1 and 10:1. In some embodiments, the ratio between Δ9-THC combined with THCa and CBN combined with CBNa is between 500:1 and 150:1. In some embodiments, the ratio between Δ9-THC combined with THCa and CBC combined with CBCa is between 400:1 and 50:1. In some embodiments, the ratio between Δ9-THC combined with THCa and THCV combined with THCVa is between 1000:1 and 100:1.

### BRIEF DESCRIPTION OF THE FIGURES

In some instances, the disclosure may be more completely understood in the context and consideration of the following detailed description of embodiments of the disclosure and in connection with the accompanying Figure, in which:

FIG. 1 shows a schematic illustration of the neuroprotective mechanisms of action of cannabinoids for preventing, treating, or slowing the progression of ALS.

FIG. 2 shows a schematic of an exemplary nanocarrier.

### DETAILED DESCRIPTION

The present disclosure is directed to cannabinoid-containing compositions, formulations, and methods for use thereof, for preventing, treating, and/or slowing the progression of amyotrophic lateral sclerosis (“ALS”). Unless defined otherwise, all technical and scientific terms used herein have the same meaning as commonly understood by one of ordinary skill in the art pertinent to the methods and compositions described. The definitions provided herein are to facilitate understanding of certain terms used frequently herein.

As used herein, the singular forms “a”, “an”, and “the” encompass embodiments having plural referents, unless the content clearly dictates otherwise.

As used herein, a “functional” cannabinoid product is a cannabinoid product in a form in which it exhibits a property and/or activity by which it is characterized. A functional cannabinoid product comprises one or more active component(s), e.g., Δ9-THC, and, optionally, one or more non-active, e.g., non-cannabinoid, components, e.g., β-myrcene, terpinolene, etc.

As used herein, the term “delivery” refers to one or more routes of administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “lipid nanoparticle” refers to any lipid composition that can be used to deliver a therapeutic agent (e.g., a functional cannabinoid product) including, but not limited to, liposomes, e.g., wherein an aqueous volume is encapsulated by an amphipathic lipid bilayer; or wherein the lipids coat an interior comprising a large molecular component with a reduced aqueous interior; or lipid aggregates or micelles, wherein the encapsulated component is contained within a relatively disordered lipid mixture. See, e.g., Akbarzadehl et al., Nanoscale Research Letters 8:102 (2013).

The term “liposome” refers to microscopic lipid vesicles composed of a bilayer of phospholipids or any similar amphipathic lipids encapsulating an internal aqueous medium. Bozzuto and Molinari, International Journal of Nanomedicine 10:975-999 (2015). Liposomes of the present disclosure can be unilamellar vesicles such as small unilamellar vesicles (SUVs) and large unilamellar vesicles (LUVs), and smaller multilamellar vesicles (MLV), typically varying in size, e.g., from 50 nm to 500 nm. No particular limitation is imposed on the liposomal membrane structure in the present disclosure. The term liposomal membrane refers to the bilayer of phospholipids separating the internal aqueous medium from the external aqueous medium.

Exemplary liposomal membranes useful in the current disclosure may be formed from a variety of vesicle-forming lipids, typically including dialiphatic chain lipids, such as phospholipids, diglycerides, dialiphatic glycolipids, egg sphingomyelin and glycosphingolipid, cholesterol, and derivatives thereof, and combinations thereof. Phospholipids are amphiphilic agents having hydrophobic groups formed of long-chain alkyl chains, and a hydrophilic group containing a phosphate moiety. The group of phospholipids includes phosphatidic acid, phosphatidyl glycerols, phosphatidylcholines, phosphatidylethanolamines, phosphatidylinositols, phosphatidylserines, and mixtures thereof. In some embodiments, the phospholipids are chosen from egg yolk phosphatidylcholine (EYPC), soy phosphatidylcholine (SPC), palmitoyl-oleoyl phosphatidylcholine, dioleyl phosphatidylcholine, 1,2-dipalmitoyl-sn-glycero-3-phosphocholine (DPPC), 1,2-dimyristoyl-sn-phosphatidylcholine (DMPC), hydrogenated soy phosphatidylcholine (HSPC), distearoyl phosphatidylcholine (DSPC), or hydrogenated egg yolk phosphatidylcholine (HEPC), egg phosphatidylglycerol, distearoylphosphatidylglycerol (DSPG), sterol modified lipids, cationic lipids and zwitterlipids.

Liposomes can be prepared by any of the techniques known in the art. See, e.g., Shah et al., Journal of Controlled Release 253:37-45 (2017). For example, the liposomes can be formed by the conventional technique for preparing multilamellar lipid vesicles (MLVs), that is, by depositing one or more selected lipids on the inside walls of a suitable vessel by dissolving the lipids in chloroform and then evaporating the chloroform, and by then adding the aqueous solution which is to be encapsulated to the vessel, allowing the aqueous solution to hydrate the lipid, and swirling or vortexing the resulting lipid suspension. This process engenders a mixture including the desired liposomes. Alternatively, techniques used for producing large unilamellar lipid vesicles (LUVs), such as reverse-phase evaporation, infusion procedures, and detergent dilution, can be used to produce the liposomes. A review of these and other methods for producing lipid vesicles can be found in: Liposome Technology: Liposome preparation and related Techniques, 3rd addition, 2006, G. Gregoriadis, ed.). For example, the lipid-containing particles can be in the form of steroidal lipid vesicles, stable plurilamellar lipid vesicles (SPLVs), monophasic vesicles (MPVs), or lipid matrix carriers (LMCs). In the case of MLVs, if desired, the liposomes can be subjected to multiple (five or more) freeze-thaw cycles to enhance their trapped volumes and trapping efficiencies and to provide a more uniform interlamellar distribution of solute.

Following liposome preparation, the liposomes are optionally sized to achieve a desired size range and relatively narrow distribution of liposome sizes. A size range of from about 30 to about 200 nanometers allows the liposome suspension to be sterilized by filtration through a conventional sterile filter, typically a 0.22 micron or 0.4 micron filter. The filter sterilization method can be carried out on a high throughput basis if the liposomes have been sized down to about 20-300 nanometers. Several techniques are available for sizing liposomes to a desired size. Sonicating a liposome suspension either by bath or probe sonication produces a progressive size reduction down to small unilamellar vesicles less than about 50 nanometer in size. Homogenization is another method which relies on shearing energy to fragment large liposomes into smaller ones. In a typical homogenization procedure, multilamellar vesicles are recirculated through a standard emulsion homogenizer until selected liposome sizes, typically between about 50 and 500 nanometers, are observed. In both methods, the particle size distribution can be monitored by conventional laser-beam particle size determination. Extrusion of liposome through a small-pore polycarbonate membrane or an asymmetric ceramic membrane is also an effective method for reducing liposome sizes to a relatively well-defined size distribution. Typically, the suspension is cycled through the membrane one or more times until the desired liposome size distribution is achieved. The liposomes may be extruded through successively smaller-pore membranes, to achieve a gradual reduction in liposome size. Other useful sizing methods such as sonication, solvent vaporization or reverse phase evaporation are known to those of skill in the art.

Exemplary liposomes for use in various embodiments of the disclosure have a size of from about 30 nm to about 300 nm, e.g., from about 50 nm to about 250 nm.

The internal aqueous medium, as referred to herein, typically is the original medium in which the liposomes were prepared and which initially becomes encapsulated upon formation of the liposome. In accordance with the present disclosure, freshly prepared liposomes encapsulating the original aqueous medium can be used directly for active loading. Embodiments are also envisaged however wherein the liposomes, after preparation, are dehydrated, e.g. for storage. In such embodiments the present process may involve addition of the dehydrated liposomes directly to the external aqueous medium used to create the transmembrane gradients. However it is also possible to hydrate the liposomes in another external medium first, as will be understood by those skilled in the art. Liposomes are optionally dehydrated under reduced pressure using standard freeze-drying equipment or equivalent apparatus. In various embodiments, the liposomes and their surrounding medium are frozen in liquid nitrogen before being dehydrated and placed under reduced pressure. To ensure that the liposomes will survive the dehydration process without losing a substantial portion of their internal contents, one or more protective sugars are typically employed to interact with the lipid vesicle membranes and keep them intact as the water in the system is removed. A variety of sugars can be used, including such sugars as trehalose, maltose, sucrose, glucose, lactose, and dextran. In general, disaccharide sugars have been found to work better than monosaccharide sugars, with the disaccharide sugars trehalose and sucrose being most effective. Typically, one or more sugars are included as part of either the internal or external media of the lipid vesicles. Most preferably, the sugars are included in both the internal and external media so that they can interact with both the inside and outside surfaces of the liposomes' membranes. Inclusion in the internal medium is accomplished by adding the sugar or sugars to the buffer which becomes encapsulated in the lipid vesicles during the liposome formation process. In addition to the sugars, a co-lyophilization agent such as glycine, betaine or carnitine, can be included to further increase the stability of the lyophilized liposome chelators. In these embodiments the external medium used during the active loading process should also preferably include one or more of the protective sugars

As is generally known to those skilled in the art, polyethylene glycol (PEG)-lipid conjugates have been used extensively to improve circulation times for liposome-encapsulated functional compounds, to avoid or reduce premature leakage of the functional compound from the liposomal composition and to avoid detection of liposomes by the body's immune system. Attachment of PEG-derived lipids onto liposomes is called PEGylation. Hence, in one embodiment of the disclosure, the liposomes are PEGylated liposomes. Suitable PEG-derived lipids according to the disclosure, include conjugates of DSPE-PEG, functionalized with one of carboxylic acids, glutathione (GSH), maleimides (MAL), 3-(2-pyridyldithio) propionic acid (PDP), cyanur, azides, amines, biotin or folate, in which the molecular weight of PEG is between 2000 and 5000 g/mol. Other suitable PEG-derived lipids are mPEGs conjugated with ceramide, having either C8- or C 16-tails, in which the molecular weight of mPEG is between 750 and 5000 daltons. Still other appropriate ligands are mPEGs or functionalized PEGs conjugated with glycerophospholipds like 1,2-dimyristoyl-sn-glycero-3-phosphoethanolamine (DMPE), 1,2-dipalmitoyl-sn-glycero-3-phosphoethanolamine (DPPE), 1,2-dioleoyl-sn-glycero-3-phosphoethanolamine (DOPE) and 1,2-distearoyl-sn-glycero-3-phosphoethanolamine (DSPE), and the like. PEGylation of liposomes is a technique generally known by those skilled in the art.

In various embodiments, the liposomes are PEGylated with DSPE-mPEG conjugates (wherein the molecular weight of PEG is typically within the range of 750-5000 daltons, e.g. 2000 daltons). The phospholipid composition of an exemplary PEGylated lipsome of the disclosure may comprise up to, e.g., 0.8-20 mol % of PEG-lipid conjugates.

As used herein, “lipid encapsulated” or “lipid encapsulation” can refer to a lipid formulation which provides a compound (e.g., a functional cannabinoid product) with full encapsulation, partial encapsulation, or both.

As used herein the term “treating” refers to the administration of a treatment that eliminates, alleviates, inhibits or slows the progression of, or reverses progression of, in part or in whole, any one or more of the pathological hallmarks or symptoms of any one of the diseases and disorders being treated. Such diseases include, but are not limited to ALS. For example, the cannabinoid-containing compositions described herein may also prove efficacious in treating Parkinson's and other neurodegenerative and autoimmune diseases.

The phrase “therapeutically effective” as used herein is intended to qualify the amount of a composition useful to treat a disease or disorder, or the combined amount of active ingredients in the case of combination therapy. This amount, or combined amount, will achieve the goal of preventing, treating, and/or slowing progression of the condition.

As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human. A subject can be a patient, which refers to a human presenting to a medical provider for diagnosis or treatment of a disease. The term “subject” is used herein interchangeably with “individual” or “patient.” A subject can be afflicted with or is susceptible to a disease, disorder, or condition but may or may not display symptoms of the disease, disorder, or condition.

### Mechanism of Action

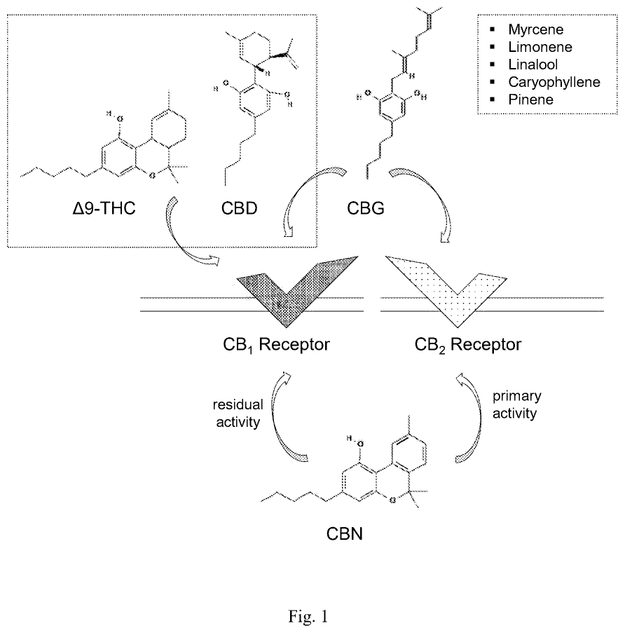

FIG. 1 shows a mechanism of action for cannabinoids with respect to treating the effects of ALS. This mechanism is based in part on the ability of Δ9-THC, alone or in combination with other cannabinoids and non-cannabinoids, to modulate and/or attenuate oxidative stress via its anti-glutamatergic and anti-oxidant activity. See, e.g., Raman et al. Amyotrophic lateral sclerosis: delayed disease progression in mice by treatment with a cannabinoid. Amyotroph Lateral Scler Other Motor Neuron Disord. 2004; 5(1):33-9. This mechanism is also based on the ability of Δ9-THC, alone or in combination with other cannabinoids and non-cannabinoids, to protect against excitotoxicity. Raman et al. This mechanism is further based on evidence that Δ9-THC and CBD in approximately a ratio of 1:1 has neuroprotective effects. Moreno-Martet M et al. This mechanism of action is also based on the activity of other cannabinoids, including but not limited to CBN, CBG, THCV, with respect to the CB₁/CB₂ receptors where such activity has neuroprotective effects due to decreases in microglial activation, macrophage activation, release of presynaptic glutamate, and/or formation of ROS. Velayudhan et al. This is consistent with evidence that CBN and CBC demonstrate potent anti-inflammatory effects. De Petrocellis L, Ligresti A, Moriello A S, et al. Effects of cannabinoids and cannabinoid-enriched Cannabis extracts on TRP channels and endocannabinoid metabolic enzymes. Br J Pharmacol. 2011; 163(7):1479-94.

This mechanism of action further contemplates the synergistic effects of non-cannabinoids, such as terpenes. Russo E B. Taming THC: potential Cannabis synergy and phytocannabinoid-terpenoid entourage effects. Br J Pharmacol. 2011; 163(7):1344-64. Certain classes of terpenes have been associated with synergistic or enhanced therapeutic effects that may be relevant to preventing, treating, and/or slowing of the progression of ALS when they accompany functional cannabinoids, for example: Myrcene (having synergy with CBN and β-Myrcene associated with inflammation blocking via PGE-2 pathway); Limonene (having synergy with Δ9-THC, CBD, CBC, CBG, and CBN and associated with immunostimulation); Linalool (having synergy with Δ9-THC, CBD, CBG, THCV, CBDV, and CBN and associated with anticonvulsant and anti-glutamate activity); Caryophyllene (having synergy with Δ9-THC, CBD, CBC, and CBG), Nerolidol (having synergy with Δ9-THC and CBN); and Pinene (having synergy with Δ9-THC, CBD, CBG, and CBN and α-Pinene associated with anti-inflammatory activity and acetylcholinesterase inhibition).

The potential therapeutic effects of the mechanism of action shown in FIG. 1 include delay of disease onset, improvement in motor impairment, and increase of survival in the case of ALS. See, e.g., Giacoppo S et al. Can cannabinoids be a potential therapeutic tool in amyotrophic lateral sclerosis?.Neural Regen Res. 2016; 11(12):1896-1899.

This mechanism may also involve cannabinoids including (−)-trans-Δ9-tetrahydrocannabiphorol (“Δ9-THCP”) and cannabidiphorol (“CBDP”), which are Δ9-THC and CBD homologs respectively. The cannabinoid Δ9-THCP is thought to have higher binding affinity for the CB₁ receptor as well as greater cannabimimetic activity than Δ9-THC. Citti et al. A novel phytocannabinoid isolated from Cannabis sativa L. with an in vivo cannabimimetic activity higher than Δ-tetrahydrocannabinol: Δ-Tetrahydrocannabiphorol. Sci Rep. 2019; 9(1):20335. CBDP is also thought to have anti-inflammatory and anti-oxidant effects similar to those of CBD. Δ9-THCP and CBDP are implicated by the mechanism of action illustrated in FIG. 1 as potential substitutes for or supplements to Δ9-THC and CBD.

### Cannabis Extracts

As illustrated in FIG. 1, cannabinoids have potential for preventing, treating, and/or slowing the progression of ALS. This is due to the neuroprotective effects that result from the antioxidant, anti-inflammatory, and/or anti-excitotoxic properties exhibited by these compounds. Cannabinoids can be extracted from the Cannabis plant in isolate form as well as with a broader spectrum of other Cannabis-derived compounds, including terpenes.

The spectrum and relative concentrations of Cannabis-derived compounds associated with a given extract can depend on the starting material from which Cannabis-derived compounds are extracted, the method of extraction, and any processing or isolation performed. The starting material in turn can depend upon the characteristics of the Cannabis strain and its associated biochemical profile. Cannabis strains can be bred and/or engineered, genetically or otherwise, to produce specific cannabinoid and non-cannabinoid profiles. Through the cross-breeding and/or engineering of Cannabis strains, it is possible to cultivate a starting material that includes the desired profile of cannabinoids and non-cannabinoids. Extraction techniques can be employed that yield the desired profile of cannabinoid and non-cannabinoids. Further processing and/or isolation steps can be employed to refine the extract to reflect the desired profile of cannabinoid and non-cannabinoids for a given application.

Cannabinoid-containing compositions can also be formulated or otherwise constituted to possess specific cannabinoid and/or non-cannabinoid profiles. For example, cannabinoids and/or non-cannabinoids can be combined to form a compound that possesses a particular biochemical profile. Multiple Cannabis extracts can also be combined. Cannabis-derived compositions can also be fortified or enriched with additional cannabinoids, non-cannabinoids, and/or one or more active or non-active components to enhance, refine, and/or modify a given property. Pharmaceutical compositions prepared according to the present disclosure may be formulated from pure cannabinoids or in combination with carriers and excipients, which are well-known to those skilled in the art and/or are described herein.

In the case of Cannabis extracts and cannabinoid-containing compositions for use in preventing, treating, and/or slowing the progression of ALS, the combined presence of Δ9-THC and CBD has been shown to have therapeutic benefits owing to the potential for up-regulation of CB₂ receptors, together with an increase of the NAPE-PLD enzyme. Applicants have identified additional cannabinoid and non-cannabinoid compounds that can be co-extracted and/or formulated with Δ9-THC and CBD to provide benefits to ALS patients. The synergistic and additive effects of these additional compounds may be further enhanced by use of pharmaceutical formulations and delivery methods described herein. The therapeutic synergy that can occur when a spectrum of Cannabis-derived compounds is administered to a patient has been referred to in the literature as the “entourage effect.” Ben-Shabat S, An entourage effect: inactive endogenous fatty acid glycerol esters enhance 2-arachidonoyl-glycerol cannabinoid activity. Eur J Pharmacol. 1998 Jul. 17; 353(1):23-31.

Cannabinoid-containing pharmaceutical compositions in accordance with the present disclosure may include a functional cannabinoid product that comprises one or more active component(s). Consistent with the mechanism of action shown in FIG. 1, there may be one or more active component(s) associated with a therapeutic effect in the context of preventing, treating, and/or slowing the progression of ALS. An active component may be one or more cannabinoids, or one or more non-cannabinoids, or a combination thereof. An active component may have activity alone or in combination with an anticancer drug, immunomodulatory drug, stem cell differentiation biomolecule, or cell activity modulator.

There are various techniques and technologies available for extracting Cannabis-derivatives, including cannabinoids, from the Cannabis plant. Such extraction techniques include but are not limited to: supercritical or subcritical extraction with CO₂, hot gas extraction, extraction with solvents, hydrocarbon extraction, and cryogenic ethanol extraction. A particular extraction method can be selected depending on, for example, the application, the need to remove impurities, the need to isolate specific compounds, and/or the need to extract compounds without upsetting volatile elements of the Cannabis plant. In some embodiments, cryogenic ethanol extraction is utilized. In such embodiments, frozen and size-reduced Cannabis plant material is charged into an extractor. The plant material is agitated and recirculated until extraction is complete. A recirculation pump collects the Cannabis-derivative miscella in a tank where it can then be removed for further processing.

### Formulations

For preparing pharmaceutical compositions from Cannabis extracts and/or Cannabis derivatives in accordance with the present disclosure, pharmaceutically acceptable carriers can be either solid or liquid. Solid form preparations include, but are not limited to, powders, tablets, pills, capsules, cachets, suppositories, and dispersible granules. A solid carrier may be one or more substances, which may act as diluents, flavoring agents, binders, preservatives, tablet disintegrating agents, or encapsulating material. The compositions of the present disclosure can be formulated by processes which include processes that are the same or analogous to those known in the chemical arts in light of the description herein.

The pharmaceutical composition is preferably in unit dosage form. In such form, the preparation is subdivided into unit doses containing appropriate quantities of a functional cannabinoid product comprising one or more active component(s). The unit dosage form can be a packaged preparation, the package containing discrete quantities of preparation, such as packeted tablets, capsules, and powders in vials or ampoules. The unit dosage form of the pharmaceutical composition can also be a capsule, tablet, cachet, or lozenge, or it can be the appropriate number of any of these in packaged form.

The quantity of the functional cannabinoid product comprising one or more active component(s) in a unit dose preparation may be varied or adjusted, for example from about 0.1 mg/kg to about 200 mg/kg, preferably from about 0.5 mg/kg to about 100 mg/kg, according to the application and the potency of the one or more active component(s).

The pharmaceutical compositions disclosed herein can, if desired, contain other compatible therapeutic agents. For example, the cannabinoid-containing composition as described herein can be formulated into a pharmaceutical composition that includes a second active ingredient such as an anticancer drug, immunomodulatory drug, stem cell differentiation biomolecule, or cell activity modulator. These secondary therapeutic agents can also be co-administered with the presently described cannabinoid-containing compositions. Such co-administration could include sequential administration.

Determination of the proper dosage for a particular situation is within the skill of the art. Determination of a therapeutically effective dosage is typically based on animal model studies followed by human clinical trials and is guided by determining effective dosages and administration protocols that significantly reduce the occurrence or severity of the subject disease or condition in model subjects. Effective doses of the compositions of the present disclosure vary depending upon different factors, including means of administration, target site, physiological state of the patient, whether the patient is human or an animal, other medications administered, as well as the specific activity of the composition itself and its ability to elicit the desired response in the individual.

Usually, the patient is a human, but in some diseases, the patient can be a non-human mammal. Typically, dosage regimens are adjusted to provide an optimum therapeutic response, i.e., to optimize safety and efficacy. Thus, a therapeutically effective amount is also one in which any undesired collateral effects are outweighed by beneficial effects of administering a composition. Generally, treatment is initiated with smaller dosages, which are less than the optimum dose of the compound. Thereafter, the dosage is increased by small increments until the optimum effect under the circumstances is reached.

Effective dosages can be achieved by single or multiple administrations, including, e.g., multiple administrations per day or daily, weekly, bi-weekly, or monthly adminitrations. For example, a total daily dosage may be divided and administered in portions during the day, if desired. In certain variations, a regimen consists of an initial administration followed by multiple, subsequent administrations at semi-weekly, weekly, or bi-weekly intervals. Another regimen consists of an initial administration followed by multiple, subsequent administrations at monthly or bi-monthly intervals. Alternatively, administrations can be performed on an irregular basis as indicated by monitoring of clinical symptoms and/or physiological correlates of the disorder, disease, or condition.

The controlled-release characteristics of the formulation may be varied by modifying the relative amounts of excipients included into the formulation, for example, the amounts of emulsifying agents and viscolising agents, if present. The Hydrophilic Lipophilic Balance (“HLB”) system, the balance between the hydrophilic and lipophilic moieties of a surface-active molecule, is a basis for selecting emulsifying agents. The controlled-release characteristics may therefore be varied to suit the type of functional cannabinoid product included in the formulation, since it may be desirable that the formulation remain in contact with the mucosal surface for a period of time sufficient to allow substantially all of the functional cannabinoid product to be absorbed through the mucosal surface into the systemic circulation. The rate at which the functional cannabinoid product is absorbed is dependent upon the the nature of the functional cannabinoid product. In the case of cannabinoids, significant absorption through the buccal or sublingual mucosa is achieved in a period of about 10 minutes. It is therefore desirable that any formulation for delivery of cannabinoids remain substantially intact and in contact with the mucosal surface for at least this time.

In some embodiments, where the dosage form is placed in contact with saliva, the following pharmaceutically acceptable excipients may be included to give a suitable degree of viscosity to the cannabinoid-containing formulation: acacia, cetostearyl, cetyl, anionic emulsifying wax, cellulose, diethanolamine, gelatin, glyceryl monoleate, glyceryl monostearate, lecithin, medium chain triglycerides, methylcellulose, nonionic emulsifying wax, poloxamer, polydextrose, polyethoxylated castor oil, polyoxyethylene alkyl, ethers, polyoxyethylene ethers, polyoxyethelene stearates, pregelatinized starch, propylene glycol alginate, sodium lauryl sulfate, sorbitan esters, starch, tri-sodium citrate.

In some embodiments, formulations prepared according to the present disclosure will disintegrate completely within a period of from 0.1 to 60 minutes, more preferably within 0.5 to 15 minutes, however, formulations within the scope of the present disclosure can be produced in which the disintegration time is at least 90 minutes.

In some embodiments, the effective daily dose of the functional cannabinoid product is between 5 mg and 1000 mg. In some embodiments, the effective daily dose of a functional cannabinoid product is between 5 mg and 500 mg. In some embodiments, the effective daily dose of a functional cannabinoid product is between 5 mg and 100 mg.

An example of a pharmaceutical composition of the present disclosure includes a functional cannabinoid product where the functional cannabinoid product comprises at least 95% cannabinoids including one or more of Δ9-THC, THCa, THCV, THCVa, CBD, CBDa, CBN, CBNa, CBG, CBGa, CBC, CBCa and at least 2% non-cannabinoids including one or more of β-Myrcene, δ-Limonene, Linalool, β-Caryophyllene, α-Humulene, β-Nerolidol, (−)-Guaiol, (−)-α-Bisabolol, Terpinolene, Nerolidol, Guaiol, Geraniol, Ocimene 2, Endo-Fenchyl Alcohol, α-Terpineol, Geranyl Acetate, cis-Nerolidol, α-Pinene, (−)-β-Pinene, Farnesene, Valencene, trans-Nerolidol, Caryophyllene Oxide.

An example of a pharmaceutical composition of the present disclosure includes a functional cannabinoid product where the functional cannabinoid product comprises at least 85% cannabinoids including one or more of Δ9-THC, THCa, THCV, THCVa, CBD, CBDa, CBN, CBNa, CBG, CBGa, CBC, CBCa and at least 10% non-cannabinoids including one or more of β-Myrcene, δ-Limonene, Linalool, β-Caryophyllene, α-Humulene, β-Nerolidol, (−)-Guaiol, (−)-α-Bisabolol, Terpinolene, Nerolidol, Guaiol, Geraniol, Ocimene 2, Endo-Fenchyl Alcohol, α-Terpineol, Geranyl Acetate, cis-Nerolidol, α-Pinene, (−)-β-Pinene, Farnesene, Valencene, trans-Nerolidol, Caryophyllene Oxide.

An exemplary pharmaceutical composition includes a functional cannabinoid product where the ratio between Δ9-THC combined with THCa and CBD combined with CBDa is between 100:1 and 1:1. Other examples include pharmaceutical compositions where the ratio between Δ9-THC combined with THCa and CBD combined with CBDa is 50:1 and 1:1. Other examples include pharmaceutical compositions where the ratio between Δ9-THC combined with THCa and CBD combined with CBDa is between 20:1 and 1:1. Other examples include pharmaceutical compositions where the ratio between Δ9-THC combined with THCa and CBD combined with CBDa is between 30:1 and 10:1. Other examples include pharmaceutical compositions where the ratio between Δ9-THC combined with THCa and CBD combined with CBDa is between 25:1 and 15:1.

An exemplary pharmaceutical composition includes a functional cannabinoid product where the ratio between Δ9-THC combined with THCa and CBG combined with CBGa is between 100:1 and 1:1. Other examples include pharmaceutical compositions where the ratio between Δ9-THC combined with THCa and CBG combined with CBGa is 60:1 and 1:1. Other examples include pharmaceutical compositions where the ratio between Δ9-THC combined with THCa and CBG combined with CBGa is between 20:1 and 1:1. Other examples include pharmaceutical compositions where the ratio between Δ9-THC combined with THCa and CBG combined with CBGa is between 30:1 and 10:1. Other examples include pharmaceutical compositions where the ratio between Δ9-THC combined with THCa and CBG combined with CBGa is between 25:1 and 15:1.

An exemplary pharmaceutical composition includes a functional cannabinoid product where the ratio between Δ9-THC combined with THCa and CBN combined with CBNa is between 500:1 and 1:1. Other examples include pharmaceutical compositions where the ratio between Δ9-THC combined with THCa and CBN combined with CBNa is 500:1 and 150:1. Other examples include pharmaceutical compositions where the ratio between Δ9-THC combined with THCa and CBN combined with CBNa is between 20:1 and 1:1. Other examples include pharmaceutical compositions where the ratio between Δ9-THC combined with THCa and CBN combined with CBNa is between 30:1 and 10:1. Other examples include pharmaceutical compositions where the ratio between Δ9-THC combined with THCa and CBN combined with CBNa is between 25:1 and 15:1.

An exemplary pharmaceutical composition includes a functional cannabinoid product where the ratio between Δ9-THC combined with THCa and CBC combined with CBCa is between 400:1 and 1:1. Other examples include pharmaceutical compositions where the ratio between Δ9-THC combined with THCa and CBC combined with CBCa is 400:1 and 50:1. Other examples include pharmaceutical compositions where the ratio between Δ9-THC combined with THCa and CBC combined with CBCa is between 20:1 and 1:1. Other examples include pharmaceutical compositions where the ratio between Δ9-THC combined with THCa and CBC combined with CBCa is between 30:1 and 10:1. Other examples include pharmaceutical compositions where the ratio between Δ9-THC combined with THCa and CBC combined with CBCa is between 25:1 and 15:1.

An exemplary pharmaceutical composition includes a functional cannabinoid product where the ratio between Δ9-THC combined with THCa and THCV combined with THCVa is between 1000:1 and 1:1. Other examples include pharmaceutical compositions where the ratio between Δ9-THC combined with THCa and THCV combined with THCVa is 1000:1 and 100:1. Other examples include pharmaceutical compositions where the ratio between Δ9-THC combined with THCa and THCV combined with THCVa is between 20:1 and 1:1. Other examples include pharmaceutical compositions where the ratio between 49-THC combined with THCa and THCV combined with THCVa is between 30:1 and 10:1. Other examples include pharmaceutical compositions where the ratio between Δ9-THC combined with THCa and THCV combined with THCVa is between 25:1 and 15:1.

### EXAMPLES

Exemplary functional cannabinoid products for use in the preparation of various pharmaceutical compositions and formulations of the present disclosure include:

### Example 1

A functional cannabinoid product comprising one or more cannabinoids, wherein the percentage (%) of mass and mass in milligrams for each is as follows:

TABLE 1.1

| Cannabinoid | Mass (%) | Mass (mg) |
|---|---|---|
| Δ9-THC | 20.84% | 040.60 |
| THCa | 60.78% | 118.40 |
| CBD | 02.21% | 004.30 |
| CBDa | 09.70% | 018.90 |
| CBG | 00.51% | 001.00 |
| CBGa | 02.52% | 004.90 |
| CBN | 00.41% | 000.80 |
| CBC | 00.51% | 001.00 |
| Total Cannabinoid Content | 97.48% | 189.9 |

A functional cannabinoid product comprising one or more non-cannabinoids, wherein the percentage (%) of mass and mass in milligrams for each is as follows:

TABLE 1.2

| Non-Cannabinoid | Mass (%) | Mass (mg) |
|---|---|---|
| β-Myrcene | 00.15% | 000.30 |
| δ-Limonene | 00.15% | 000.30 |
| Linalool | 00.15% | 000.30 |
| β-Caryophyllene | 00.82% | 001.60 |
| α-Humulene | 00.26% | 000.50 |
| β-Nerolidol | 00.21% | 000.40 |
| (−)-Guaiol | 00.36% | 000.70 |
| (−)-α-Bisabolol | 00.41% | 000.80 |
| Total Terpene Content | 2.51% | 4.9 |

### Example 2

A functional cannabinoid product comprising one or more cannabinoids, wherein the percentage (%) of mass and mass in milligrams for each is as follows:

TABLE 2.1

| Cannabinoid | Mass (%) | Mass (mg) |
|---|---|---|
| Δ9-THC | 93.20% | 223.50 |
| THCV | 00.67% | 001.60 |
| CBG | 01.79% | 004.30 |
| CBN | 00.83% | 002.00 |
| CBC | 01.42% | 003.40 |
| Total Cannabinoid Content | 97.91% | 234.8 |

A functional cannabinoid product comprising one or more non-cannabinoids, wherein the percentage (%) of mass and mass in milligrams for each is as follows:

TABLE 2.2

| Non-Cannabinoid | Mass (%) | Mass (mg) |
|---|---|---|
| β-Myrcene | 00.58% | 001.40 |
| Terpinolene | 00.46% | 001.10 |
| Nerolidol | 00.33% | 000.80 |
| β-Caryophyllene | 00.17% | 000.40 |
| Guaiol | 00.17% | 000.40 |
| Geraniol | 00.13% | 000.30 |
| α-Humulene | 00.08% | 000.20 |
| Linalool | 00.08% | 000.20 |
| (−)-α-Bisabolol | 00.08% | 000.20 |
| Total Terpene Content | 2.08% | 5 |

### Example 3

A functional cannabinoid product comprising one or more cannabinoids, wherein the percentage (%) of mass and mass in milligrams for each is as follows:

TABLE 3.1

| Cannabinoid | Mass (%) | Mass (mg) |
|---|---|---|
| Δ9-THC | 14.12% | 035.90 |
| THCa | 76.13% | 193.60 |
| THCV | 00.12% | 000.30 |
| CBDa | 00.20% | 000.50 |
| CBG | 00.67% | 001.70 |
| CBGa | 04.40% | 011.20 |
| CBN | 00.24% | 000.60 |
| CBC | 00.24% | 000.60 |
| Total Cannabinoid Content | 96.12% | 244.4 |

A functional cannabinoid product comprising one or more non-cannabinoids, wherein the percentage (%) of mass and mass in milligrams for each is as follows:

TABLE 3.2

| Non-Cannabinoid | Mass (%) | Mass (mg) |
|---|---|---|
| β-Myrcene | 00.12% | 000.30 |
| δ-Limonene | 00.16% | 000.40 |
| Ocimene 2 | 00.16% | 000.40 |
| Terpinolene | 00.31% | 000.80 |
| Linalool | 00.12% | 000.30 |
| Endo-Fenchyl Alcohol | 00.12% | 000.30 |
| α-Terpineol | 00.24% | 000.60 |
| β-Caryophyllene | 00.35% | 000.90 |
| α-Humulene | 00.12% | 000.30 |
| Geranyl Acetate | 00.59% | 001.50 |
| cis-Nerolidol | 00.12% | 000.30 |
| β-Nerolidol | 01.14% | 002.90 |
| (−)-Guaiol | 00.24% | 000.60 |
| (−)-α-Bisabolol | 00.12% | 000.30 |
| Total Terpene Content | 3.91% | 9.9 |

### Example 4

A functional cannabinoid product comprising one or more cannabinoids, wherein the percentage (%) of mass and mass in milligrams for each is as follows:

TABLE 4.1

| Cannabinoid | Mass (%) | Mass (mg) |
|---|---|---|
| Δ9-THC | 16.16% | 049.20 |
| THCa | 64.59% | 196.60 |
| THCV | 00.10% | 000.30 |
| CBDa | 00.20% | 000.60 |
| CBG | 00.79% | 002.40 |
| CBGa | 03.22% | 009.80 |
| CBN | 00.16% | 000.50 |
| CBC | 00.36% | 001.10 |
| Total Cannabinoid Content | 85.58% | 260.5 |

A functional cannabinoid product comprising one or more non-cannabinoids, wherein the percentage (%) of mass and mass in milligrams for each is as follows:

TABLE 4.2

| Non-Cannabinoid | Mass (%) | Mass (mg) |
|---|---|---|
| α-Pinene | 00.43% | 001.30 |
| (−) -β-Pinene | 00.23% | 000.70 |
| β-Myrcene | 02.96% | 009.00 |
| δ-Limonene | 01.05% | 003.20 |
| Ocimene 2 | 00.20% | 000.60 |
| Terpinolene | 00.10% | 000.30 |
| Linalool | 00.39% | 001.20 |
| Endo-Fenchyl Alcohol | 00.20% | 000.60 |
| α-Terpineol | 00.30% | 000.90 |
| Farnesene | 00.16% | 000.50 |
| β-Caryophyllene | 02.14% | 006.50 |
| α-Humulene | 00.59% | 001.80 |
| Valencene | 00.20% | 000.60 |
| Geranyl Acetate | 02.33% | 007.10 |
| cis-Nerolidol | 00.95% | 002.90 |
| β-Nerolidol | 01.02% | 003.10 |
| trans-Nerolidol | 00.10% | 000.30 |
| Caryophyllene Oxide | 00.13% | 000.40 |
| (−)-Guaiol | 00.10% | 000.30 |
| (−)-α-Bisabolol | 00.85% | 002.60 |
| Total Terpene Content | 14.43% | 43.9 |

### Example 5

### Liposomal Preparation

Sphingomyelin cholesterol (55:45) and sphingomyelin cholesterol DSPE-PEG (55:40:5) (PEGylated) liposomes are prepared by the thin-film hydration method. The final lipid concentration is fixed at 50 mg/mL. Lipids are solubilized in a chloroform:methanol (70:30) solution and evaporated under vacuum conditions to yield a homogeneous thin lipid film. The lipid solution is hydrated using 300 mM MgSO4 solution (pH 4). Multilamellar vesicles (MLV) are obtained by hydrating the lipid mixture in 300 mM MgSO4 at 65° C. The resulting colloidal solution of MLV's is reduced to large unilamellar vesicle (LUV) by extruding 20 times by forcing the lipid emulsion (MLV) through a mini-extruder with polycarbonate filters of 0.1 μm pore size at 60-65° C. The extruded liposomes are passed through a Sephadex G-50 column equilibrated with the external buffer SHE (300 mM sucrose, 3 mM EDTA, 20 mM HEPES) at pH 7.5 to establish the primary ion gradient. The liposomes are stored at 4° C. until drug loading is initiated. The functional cannabinoid product is loaded into the liposomes using the A23187-ionophore loading method which establishes the secondary ion gradient. Briefly, the functional cannabinoid product is dissolved at 10 mg/mL in 300 mM sucrose buffer. Both the functional cannabinoid product and the liposome suspension are pre-heated at 60° C. before being mixed together. After 15 min of incubation, EDTA (30 mM) and the A23187-ionophore (2 μg/mg of lipid) are added to the functional cannabinoid product-liposome mixture. The liposome along with the drug and ionophore are allowed to mix in a water bath at 60° C. for 60 min. The functional cannabinoid product-loaded liposome mixture is cooled in ice for 15 min. The unencapsulated functional cannabinoid product, EDTA and ionophore are removed by purification using a Sephadex G-50 column.

### Liposomal Preparation

The functional cannabinoid product incorporation in liposomes is quantitatively determined through the RP-HPLC method. The analysis is performed using a suitable C18 column in an isocratic mode with, e.g., methanol/water (70/30) containing 0.1% phosphoric acid and 1% methanol, at a flow rate of 0.5 mL/min, and an injection volume of 10 μL. Column temperature is maintained at 40° C. with a run time of 6 min. To quantify the functional cannabinoid product loaded in the liposomes, a 20× dilution in methanol is done to release drug from the liposomes and injected into the column for analysis. The encapsulation efficiency (EE %) is calculated using the formula EE %=Amount of functional cannabinoid product loaded/initial amount×100.

### Delivery Methods

Functional cannabinoid products and cannabinoid-containing pharmaceutical compositions prepared from them can be administered through a wide variety of routes of administration, including but not limited to parenteral, oral, topical, inhalation, sub-lingual, buccal, and the like. Formulations may vary according to the route of administration.

Functional cannabinoid products and pharmaceutical compositions prepared from them can be administered by inhalation, for example, intranasally and/or by inhalation of a liquidized cannabinoid-containing composition that has been vaporized, aerosolized, or nebulized. Functional cannabinoid products and pharmaceutical compositions prepared from them can be ingested or otherwise received through a feeding tube as an oral dosage form, digestive capsule, tincture, food product, or beverage. Functional cannabinoid products and pharmaceutical compositions prepared from them can be administered through sublingual or buccal intake. Functional cannabinoid products and pharmaceutical compositions prepared from them can be administered topically or transdermally, for example, through application of a transdermal patch to the skin. Functional cannabinoid products and pharmaceutical compositions prepared from them can be administered by injection, that is, intravenously, intramuscularly, intracutaneously, subcutaneously, intraduodenally, or intraperitoneally.

The complex neuroetiology and the restrictive nature of the blood-brain barrier in the context of delivering pharmaceutical agents has significantly hindered ALS treatments. The applicant has recognized significant advantages associated with nanoparticle delivery of functional cannabinoid products. For example, nanocarriers can eliminate the challenges of poor drug bioavailability in ALS as they have been proven to cross the blood-brain barrier and reach target sites while minimizing systemic side-effects. Nanoparticles are rapidly internalized by the cells and routed to the cytoplasm. The nanoparticles entering cells are mainly via endocytosis with time-, temperature- and energy-dependent manners. Various types of nanocarriers have been engineered, including solid lipid nanoparticles (“SLN”), liposomes, dendrimers, polymeric nanoparticles (“PNP”), polymeric micelles (“PM”), and virus-based nanoparticles (“VNP”). Din et al. Effective use of nanocarriers as drug delivery systems for the treatment of selected tumors. Int J Nanomedicine. 2017; 12:7291-7309.

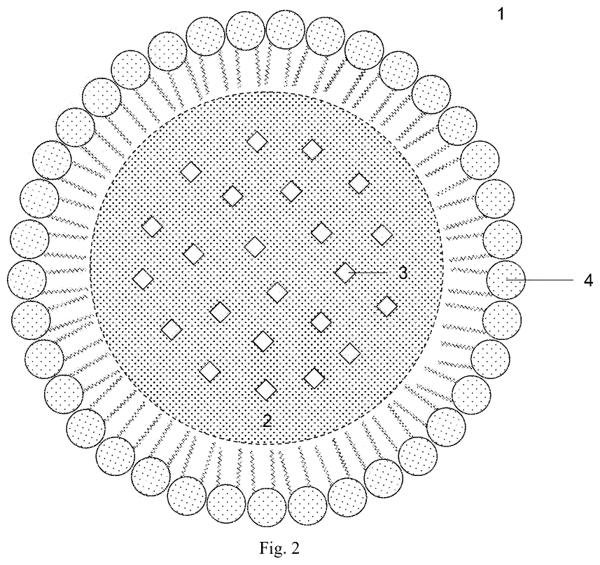

Certain aspects of the disclosure are directed to a cannabinoid-containing composition comprising: a functional cannabinoid product; and a delivery agent. In some embodiments, the delivery agent comprises a lipid nanoparticle (“LNP”). FIG. 2 shows an exemplary LNP 1 having a lipid core matrix 2 that can solubilize lipophilic molecules 3. The lipid core matrix 2 is stabilized by surfactants (emulsifiers) 4. In some embodiments, the functional cannabinoid product is encapsulated in the LNP, wherein encapsulation refers to containing a pharmaceutical composition (e.g., a functional cannabinoid product) within the interior space of the LNP. In some embodiments, the composition further comprises a pharmaceutically acceptable carrier. In some embodiments, by encapsulating the functional cannabinoid product within a delivery agent, such as a LNP, the active component(s) can be protected from an environment, which may contain enzymes or chemicals that degrade such components. LNPs typically comprise an ionizable (e.g., cationic) lipid, a non-cationic lipid (e.g., cholesterol and a phospholipid), and a PEG lipid (e.g., a conjugated PEG lipid), which can be formulated with a payload of interest, e.g., a functional cannabinoid product.

An example of a pharmaceutical composition of the present disclosure includes: the functional cannabinoid product; a pharmaceutically acceptable diluent or carrier; and/or a delivery agent. In some embodiments, the delivery agent comprises a lipid nanoparticle that encapsulates the functional cannabinoid product. In some embodiments, the delivery agent comprises a vapor that can be inhaled after the functional cannabinoid product has been nebulized or aerosolized. In such an embodiment, a pharmaceutically acceptable diluent or carrier may be included that comprises saline or a saline-like fluid. In some emodiments, the delivery agent comprises a transdermal patch that is adhered to the skin and is configured to deliver a specified dosage of the functional cannabinoid product. In some embodiments, the delivery agent comprises a tincture that administers the functional cannabinoid product sublingually. In some embodiments, the delivery agent comprises a tincture or dosage form that administers the functional cannabinoid product buccally. In some embodiments, the delivery agent comprises a liquid suitable for administration through a feeding tube, the liquid comprising the functional cannabinoid product. In some embodiments, the pharmaceutically acceptable carrier comprises a delayed or extended release tablet or capsule formulated to include the functional cannabinoid product.

### Methods of Use

Certain aspects of the present disclosure provide methods of use that are directed to preventing, treating, slowing the progression of, and/or delaying the onset of ALS by contacting an affected or susceptible cell with a composition comprising a functional cannabinoid product and a pharmaceutically acceptable diluent or carrier, as disclosed herein. When a patient is tested for or found to have a gene associated with ALS certain aspects of the present disclosure provide methods of use that are directed to delaying the onset of ALS by contacting an affected or susceptible cell with a composition comprising a functional cannabinoid product and a pharmaceutically acceptable diluent or carrier, as disclosed herein thereby preventing and/or delaying the onset of ALS. Such methods are particulary advantageous where a hereditary or familial history of ALS exists and/or in cases where genetic screening shows a predisposition towards developing ALS. Such prevention and/or delay of onset therapy may also be advantageous for patients in occupations associated with a predisposition towards developing ALS. The mechanism of action illustrated in FIG. 1 indicates that this disclosure may have therapeutic potential in the case of Parkinson's disease and other neurodegenerative and auto-immune diseases.

Certain aspects of the present disclosure provide methods of use that are directed to preventing, treating, slowing the progression of, and/or delaying the onset of ALS by contacting an affected or susceptible cell with a composition comprising a functional cannabinoid product and a pharmaceutically acceptable diluent or carrier, as disclosed herein, in conjunction with stem cell therapy for neuron re-growth and/or with targeted gene therapy for deactiving one or more of the genes associated with ALS.

The present disclosure provides the following particular embodiments:

Embodiment I. A pharmaceutical composition comprising:
a functional cannabinoid product; and
a delivery agent, the delivery agent comprising a lipid nanoparticle that encapsulates the functional cannabinoid product,
wherein the functional cannabinoid product is present in an amount that is therapeutically effective to prevent, treat, slow the progression of, or delay the onset of amyotrophic lateral sclerosis.

Embodiment II. The composition of Embodiment I, wherein the functional cannabinoid product includes one or more active components, the one or more active components comprising Δ9-THC, THCa, CBD, and CBDa, wherein the ratio of Δ9-THC combined with THCa and CBD combined with CBDa is between 20:1 and 1:1.

Embodiment III. The composition of Embodiments I or II, wherein the lipid nanoparticle is slelected from the group consisting of solid lipid nanoparticles, liposomes, lipid aggregates, and micelles.

Embodiment IV. The composition of Embodiments I-III, wherein the functional cannabinoid product and the delivery agent are combined in a tincture.

Embodiment V. The composition of Embodiments I-III, wherein the functional cannabinoid product and the delivery agent are combined in the form of a nebulized vapour.

Embodiment VI. The composition of Embodiments I-III, wherein the functional cannabinoid product and the delivery agent are combined in a transdermal patch.

Embodiment VII. A method for preventing, treating, or slowing the progression of amyotrophic lateral sclerosis comprising administering to a subject in need thereof a therapeutically effective amount of a composition of any one of Embodiments I-III.

Embodiment VIII. The method of Embodiment VII, wherein the composition is administered sublingually.

Embodiment IX. The method of Embodiment VII, wherein the composition is administered by inhalation.

Embodiment X. The method of Embodiment VII, wherein the composition is administered transdermally.

The implementations described above and other implementations are within the scope of the following claims. One skilled in the art will appreciate that the present disclosure can be practiced with embodiments other than those disclosed. The disclosed embodiments herein are presented for purposes of illustration and not limitation.

We claim:

1. A pharmaceutical composition comprising:
a functional cannabinoid product; and
a delivery agent, the delivery agent comprising a lipid nanoparticle that encapsulates the functional cannabinoid product.

2. The composition of claim 1, wherein the functional cannabinoid product is present in an amount that is therapeutically effective to prevent, treat, slow the progression of, or delay the onset of amyotrophic lateral sclerosis.

3. The composition of claim 1, wherein the functional cannabinoid product comprises Δ9-THC.

4. The composition of claim 3, wherein the functional cannabinoid product further comprises THCa, CBD, or CBDa, or a combination thereof.

5. The composition of claim 4, wherein the ratio of Δ9-THC combined with THCa, and CBD combined with CBDa is between 20:1 and 1:1

6. The composition of claim 3, wherein the functional cannabinoid product further comprises THCV, CBN, CBG, or CBC, or a combination thereof.

7. The composition of claim 3, wherein the functional cannabinoid product further comprises THCa and CBGa.

8. The composition of claim 1, wherein the functional cannabinoid product comprises the one or more cannabinoids according to Table 1.1 and, optionally, the one or more non-cannabinoids according to Table 1.2.

9. The composition of claim 1, wherein the functional cannabinoid product comprises the one or more cannabinoids according to Table 2.1 and, optionally, the one or more non-cannabinoids according to Table 2.2.

10. The composition of claim 1, wherein the functional cannabinoid product comprises the one or more cannabinoids according to Table 3.1 and, optionally, the one or more non-cannabinoids according to Table 3.2.

11. The composition of claim 1, wherein the functional cannabinoid product comprises the one or more cannabinoids according to Table 4.1 and, optionally, the one or more non-cannabinoids according to Table 4.2.

12. The composition of claim 1, wherein the lipid nanoparticle is selected from the group consisting of solid lipid nanoparticles, liposomes, lipid aggregates, and micelles.

13. The composition of claim 12, the lipid nanoparticle is a liposome.

14. The composition of claim 1, wherein the functional cannabinoid product and the delivery agent are combined in a tincture.

15. The composition of claim 1, wherein the functional cannabinoid product and the delivery agent are combined in the form of a nebulized vapour.

16. The composition of claim 1, wherein the functional cannabinoid product and the delivery agent are combined in a transdermal patch.

17. A method for preventing, treating, or slowing the progression of amyotrophic lateral sclerosis, the method comprising administering to a subject in need thereof a therapeutically effective amount of a composition claim 1.

18. The method of claim 17, wherein the composition is administered to the subject sublingually.

19. The method of claim 17, wherein the composition is administered to the subject by inhalation.

20. The method of claim 17, wherein the composition is administered to the subject transdermally.
